(19) United States
(12) Patent Application Publication
(10) Pub. No.: US 2005/0119284 A1
(43) Pub. Date: Jun. 2, 2005

(54) ANTIVIRAL AGENTS AND METHODS OF USE

(75) Inventors: Robert T. Striker, Madison, WI (US); Adnan A Elfarra, Madison, WI (US); Sjofn Gunnarsdottir, Madison, WI (US); Spencer W. Hoover, Madison, WI (US)

Correspondence Address: GODFREY & KAHN, S.C., 780 N. WATER STREET, MILWAUKEE, WI, 53202, US

(73) Assignee: Wisconsin Alumni Research Foundation -, Madison, WI (US)

(21) Appl. No.: 10/711,681

(22) Filed: Sep. 30, 2004

Related U.S. Application Data
(60) Provisional application No. 60/507,395, filed on Sep. 30, 2003.

(51) Int. Cl. A61K 31/522

(52) U.S. Cl. 514/263.3

(57) ABSTRACT

The present invention provides therapeutic methods of inhibiting RNA viruses based on the newly-discovered anti-viral activity of cis or trans-6-(2-acetylvinylthio)purine (cis-AVTP) or (trans-AVTP). RNA viruses inhibited by the methods include flaviviruses, namely hepatitis C virus (HCV) and bovine diarrhea virus (BVDV).

### RELATED APPLICATION

Present application seeks priority from a U.S. Provisional Application 60/507,395 filed on Sep. 30, 2003, which is incorporated herein by reference.

### STATEMENT AS TO RIGHTS TO INVENTIONS MADE UNDER FEDERALLY SPONSORED RESEARCH AND DEVELOPMENT

This work was supported in part by grants from the National Institutes of Health. Grant Nos. AIO55750. The Government of the United States of America may have certain rights in this invention.

### BACKGROUND

1. Field of the Invention

In general, the present invention is directed to methods of inhibiting RNA viruses. In particular, the invention provides methods of inhibiting the replication of RNA viruses, based on the newly-discovered anti-viral activity of cis or trans-6-(2-acetylvinylthio) purine (cis-AVTP or trans-AVTP).

2. Background of the Invention

Various antiviral compounds have been designed for use against virus infections in humans. However, many of these compounds are virus specific, or restricted to particular strains of a given virus. Development of compounds which are effective at treating viral diseases caused by many different viral families has only recently become a major research focus. Of the previous antiviral compounds developed, compounds which target viral replication through the use of nucleoside analogs have demonstrated the most promise in being effective against a variety of viruses. While compounds have been developed and used successfully in the treatment of DNA viruses such as herpes viruses and RNA reverse transcribing viruses like HIV, there are very few compounds which are effective against RNA viruses that do not have a DNA intermediate. Thus, there is a need in the field for novel RNA virus inhibitors, particularly inhibitors which are effective against a broad spectrum of RNA viruses.

Of all the different antiviral compounds developed, compounds which target viral replication through the use of nucleoside analogs, have demonstrated the most promise in being effective against a variety of viral diseases. Many compounds have been developed and used successfully in the treatment of DNA viruses such as herpesviruses, and RNA reverse transcribing viruses like HIV. However, there are very few compounds which are effective against RNA viruses that do not have a DNA intermediate. The reasons for this difference is and an incomplete understanding of how to exploit RNA virus replication mechanisms. RNA viruses have evolved to utilize numerous distinct replication strategies. Differences in these strategies, have demonstrated a significant enough factor that they play a key role in viral taxonomy. RNA viruses can be grouped into subgroups based on make up and orientation of their genomes. RNA viruses can contain double stranded (ds) RNA, or single stranded (ss) RNA of either positive (+) or negative (−) sense (message (m) sense or complement to message sense). Collectively +ssRNA viruses make up a majority of all known RNA viral families. Within the +ssRNA viruses, the replication strategies are further subdivided into viruses which contain a single open reading frame (ORF, ie flavivirurses) or those that contain multiple ORFs and produce smaller subgenomic RNAs (ie coronaviruses). The +ssRNA viruses also contain differences translational differences. Most viruses translate their mRNA using a 5′-methylated cap-dependent processes (ie coronaviruses). Different viruses have developed different mechanisms for capping its RNA; including virally encoded helicases, methyltransferases, and other proteins which hijack the host translation machinery. However other viruses have developed cap-independent translation strategies which utilize complex RNA structures in their 5′ non-translated regions as internal ribosomal entry sites (IRES, flaviviruses). It is possible that these key differences in virus replication and life cycle will be key determinants in drug susceptibilities, sensitivities, and development of resistance. In addition to these factors, the highly conserved motifs found in RNA virus polymerases represent another likely determinant in viral susceptibility to nucleoside analog therapeutics.

The only compound that has been approved to be used therapeutically for RNA virus infection is the guanine analog ribavirin, which may be used in conjunction with interferon-α or without interferon-α. This compound is used in the treatment of respiratory syncitial virus (RSV), lasa fever virus, and hepatitis C virus (HCV) (in conjunction with interferon-α). It is unclear how ribavirin inhibits RNA virus replication, as the different viruses it inhibits have vastly different replication strategies. Although it has demonstrated varying degrees of effectiveness for these viruses both in vivo and in vitro, the mechanism behind its antiviral properties are still largely unknown. Current research indicates that this compound, and others like it, function as RNA virus mutagens. It is believed that these compounds exploit the high evolution rate of RNA viruses, as generated by the high error rate of viral RNA-dependent RNA polymerases. The viral polymerase incorporates the nucleoside analog into the growing strand of viral genome, which then results in transitional mutations. This increase in mutagenesis is believed to drive viral replication to “error catastrophe”. However, it is still unclear why some compounds are effective for some viruses and not others.

As can be readily appreciated from the foregoing, it is therefore desirable to obtain effective inhibitors of RNA virus replication that are effective against a broad spectrum of RNA viruses.

### SUMMARY OF THE INVENTION

The inventors have recently discovered that certain thiopurine nucleoside analog compounds have antiviral activities with RNA viruses. As used herein, “RNA viruses” shall include known and yet to be identified RNA viruses proceeding through DNA intermediates as well as those lacking such intermediates. In particular, the compounds cis-AVTP and trans-AVTP, shown below as structure (I) and (II), respectively, have significant antiviral activity for RNA viruses. As well, since compounds I and II, taken alone, or in combination demonstrate ability to target glutathione rich tissues, they are known to possess reduced systemic toxicity in clinical use in comparison to previous structurally-related compounds, including previous antiviral agents. Therefore, the compounds I or II are more useful antiviral agents than previous agents as, among other things, higher viricidal doses can be achieved without the numerous side-effects observed with the previous agents.

The present provides methods for inhibiting replication of an RNA virus comprising contacting an RNA virus with a replication-inhibiting amount of a compound having the formula:

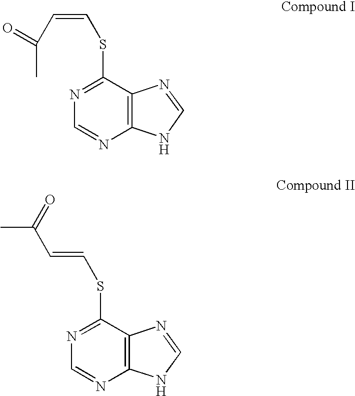

In preferred embodiments, the particular RNA virus inhibited by the present invention is a flavivirus, most preferably hepatitis C virus (HCV) or bovine diarrhea virus (BVDV). The contacting step is preferably carried out in the in vivo setting, most preferably in a glutathione-rich tissue such as liver, kidney or gastrointestinal tract tissue.

In certain embodiments, methods according to the invention utilize a derivative of compound (I) or (II). Preferably, such derivatives have the structure of virally-inhibiting metabolites of compound (I) or (II), or a combination thereof. In particular embodiments, derivatives are combined with a targeting agent that targets the derivative to a pre-selected cell or tissue. In certain embodiments, the compound further comprises a glycoside.

As well, the present provides methods for inhibiting replication of an RNA virus in a host, comprising administering to a host in need thereof a therapeutically effective amount of compound (I) or (II). The host may be any animal susceptible to RNA virus infection, preferably human, and most preferably a human liver transplant patient afflicted with an HCV infection combatable by the methods disclosed and claimed herein. The invention further encompasses the administration of therapeutically-effective derivative of compound (I) OR (II) preferably having the structure of a virally-inhibiting metabolite of compound (I) or (II).

Other objects, features and advantages of the present invention will become apparent after review of the specification, claims and drawings.

### BRIEF DESCRIPTION OF THE DRAWINGS

FIG. 1. The effect of BVDV replication in MDBK cells was measured under increasing concentrations of cis-AVTP (solid line). The antiviral effects were compared to the cytotoxic effect the drug had on cells alone (dashed line).

FIG. 2. Effect of cis-AVTP on replication of BVDV in the presence of 1 mM thymidine.

FIG. 3. The cis-AVTP metabolite 6-mercaptopurine demonstrates antiviral activity in the presence of 1 mM thymidine but 6-thioguanine does not possess antiviral activity under the same conditions. Antiviral effect of AZA is mediated through 6-mercaptopurine, but not 6-thioguanine. Viral plaque forming units, and cell numbers were determined as a function of drug equivalent thiopurines. The addition of 6-mercaptopurine (open circles, dashed line) significantly reduced the amount of virus produced in a dose dependant manner, but 6-thioguanine (open squares, dashed line) did not. Their effects on the cells though are similar.

FIG. 4: AZA has a larger specific antiviral effect than MPA on BVDV in confluent cells. Confluent MDBK cells were grown in varying concentration of AZA (circle) or MPA (square), with (open symbols) and without virus (closed symbols). After 72 hours, host cells were counted by Flow cytometry, and virus was titered by plaque assay. Due to the cytotoxicity of MPA, confluent cells (closed symbols) were exposed to doses of AZA and MPA that allowed similar amounts of cellular growth (x=0.1 for MPA and 1 for AZA). The amount of BVDV virus (open symbols) generated was significantly less in cells exposed to AZA than MPA.

FIG. 5: AZA inhibition of RNA viral replication is more profound than the inhibition of cell growth. A) MDBK cells were grown in varying concentration of AZA, with (dashed) and without virus (solid) and with (open symbols) and without (closed symbols) thymidine (T). After 72 hours, host cells were counted by Flow cytometry, and virus was titered by plaque assay. With increasing AZA, viral replication is inhibited 10-100 fold more than cell growth. In the presence of thymidine no decrease in cell survival was seen, but the majority of the antiviral effect was maintained. This shows the antiviral effect from AZA occurs in the absence of cell death. B) The absolute number of MDBK cells was decreased by AZA (closed symbols), but not if thymidine was also present (open symbols).

FIG. 6: Thiopurine Metabolism. Azathioprine is a prodrug that is metabolized to 6-mercaptopurine. 6-mercaptopurine is converted to 6-methyl mercaptopurine (MeMP) or 6-mercaptopurine riboside (MPR). In turn, MPR is converted to mono, di, and tri phosphate derivatives of thioinosine (tlM(D,T)P), methyl thioinosine (MetlM(D,T)P), and thioguanosine (tGM(D,T)P). Any of these metabolites could be responsible for the antiviral effect, and any triphosphate could potentially be incorporated into the viral genome. Only 6-thioguanosine is processed by ribonucleotide reductase into a deoxyribonucleotide (tdGTP) and incorporated into cellular DNA and this step is blocked by 1 mM thymidine. Incorporation of above thiopurine nucleotides into cellular RNA has not been observed. Metabolites of azathioprine do decrease purine synthesis through effects on glutamine-5-phosphoribosylpyrophosphate amidotransferase, but via a different mechanism, since both mycophenolate (MPA) and ribavirin decrease GTP synthesis by inhibiting inosine monophosphate dehydrogenase.

FIG. 7: Antiviral effect of AZA is equivalent or larger than equimolar amounts of ribavirin on HCV 1 bN replicon RNA level. Cells were grown in the presence of thymidine alone, or thymidine +100 uM ribavirin, or thymidine +100 uM azathioprine, and amount of HCV replicon as well as cellular GADPH mRNA was quantitated by RT-PCR. ΔCt is the difference in critical PCR threshold (HCVCₜ-GADPHCₜ), ΔΔCt is the difference of ΔCt with drug minus ΔCt without drug. Therefore larger ΔΔCt represents more inhibition of the HCV replicon.

FIG. 8: Generation and isolation of thiopurine resistant BVDV. A) Wild type BVDV plaques. B) AZA resistant BVDV mutant plaques.

FIG. 9. In vitro viral polymerase primer extension assay. Recombinant expressed HCV NS5B protein incubated with RNA template, and NTP for 0, 3, 10, 30, or 90 mins at 27° C. Transcripts were compared to 25 and 11 bp standards (M). Reactions performed with no enzyme were used as negative control (pol−).

FIG. 10 provides a comparison of cis AVTP and trans AVTP, both of which demonstrate antiviral properties.

FIG. 11 provides a graph illustrating the effects of 6 MP and its metabolites on BVDV replication.

### DETAILED DESCRIPTION OF THE INVENTION

### I. IN GENERAL

Before the present materials methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,1 95; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes l-IV (D. M. Weir and C. C. Blackwell, eds., 1986); J. H. Langenheim and K. V. Thimann, Botany: Plant Biology and Its Relation to Human Affairs (1982) John Wiley; Cell Culture and Somatic Cell Genetics of Plants, Vol. 1 (I. K. Vasil, ed. 1984); R. V. Stanier, J. L. Ingraham, M. L. Wheelis, and P. R. Painter, The Microbial World, (1986) 5th Ed., Prentice-Hall; O. D. Dhringra and J. B. Sinclair, Basic Plant Pathology Methods, (1985) CRC Press.

### II. EMBODIMENTS OF THE INVENTION

Generally, the inventors provide this present invention to enhance the understanding of how RNA viruses replicate their genome and how these mechanisms might be inhibited by different nucleoside analog compounds. Nucleoside analogs are administered as pro-drugs, which must first be converted into activated triposhphate nucleosides. The cellular enzymes which perform this task can vary from tissue to tissue and cell type to cell type, and are largely responsible for differences in activity and toxicity in different organs. It is known that ribavirin, and the thiopurine compounds AZA and 6 MP greatly affects the bone marrow, and can cause anemia and other dramatic side effects. The inventors have recently discovered that the thiopurine class of nucleoside analog compounds have antiviral activities with +sense RNA viruses. These compound have been used in the past in treatment of Crohn's disease, inflammatory bowel disease, and some tumors. These compounds are guanine analogs and have different tissue distributions, and at least one of these compounds cis-AVTP or trans AVTP has significantly few side effects. Therefore the inventors belive that these compounds may be a more useful antiviral as higher viricidal doses can be achieved with out the numerous side-effects seen with other compounds.

As a whole the following examples and embodiments contribute to the overall understanding of how anti-RNA virus nucleoside analogs function, and how the viral genetic and replicative components factor into the effectiveness of a given compound. Through these experiments the inventors will provide a greater understanding of how broad spectrum antivirals work. The inventors also demonstrate the effectiveness of two classes of antivirals against the replication of representative members of two +ssRNA viral families which have had a tremendous impact on human health and contain some of the more recent emerging infectious agents.

Accordingly, the inventors provide and teach uses of anti-viral agents through the following examples and embodiments. These examples and embodiments are for illustrative purposes only, and should not be deemed to limit the scope of the present invention.

### EXAMPLE I

Cis-AVTP as an Antiviral Agent

Generally the compound cis-6-(2-acetylvinylthio) purine (cis-AVTP), which was assayed by the inventors for its ability to inhibit RNA viruses using a bovine diarrhea virus (BVDV) model system that can be grown and manipulated in cell culture. Using this model system, the present inventors identified and measured the anti-viral effects of cis-AVTP and compared those to previously known anti-viral agents. The present invention is therefore based upon the inventors' discovery of anti-viral activity of cis-AVTP and the advantages it possesses over previous agents in terms of reduced side effects.

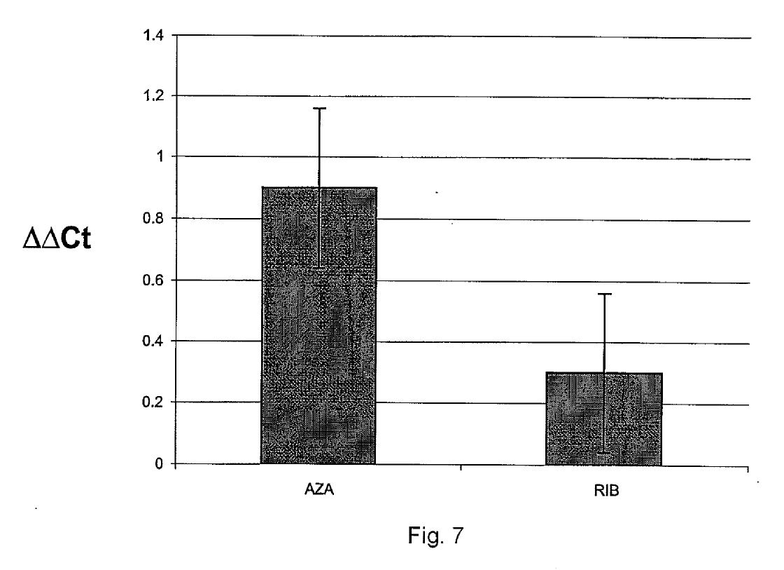

In general, the inventors have demonstrated that the thiopurines are more active against flaviviruses than ribavirin. Accordingly, the inventors demonstrated the in vitro replication of an HCV subgenomic replicon, transfected into the human hepatocyte cell line Huh-7 was more sensitive to the antiviral affects of the thiopurine azathioprine (AZA) as compared with the same concentration of ribavirin (FIG. 7) and J. R. Stangl, et al., Transplantation. (2004) vol 77, pp 54. The HCV replicon, however does not reflect a complete viral lifecycle, therefore to confirm thiopurines are effective antivirals the inventors tested the affects of AZA and two additional thiopurines 6-mercaptopurine (6MP) and 6-thioguanine (6TG). The replication of bovine viral diarrhea virus (BVDV) in Madin-Darby bovine kidney (MDBK) cells was specifically inhibited by AZA and 6MP in a dose-dependent manner (FIG. 7 herein). Interestingly 6TG, which differs from 6MP by only an amino group, and has very similar cellular effects had no antiviral affect. The inventors then tested the antiviral affects of cis-AVTP. cis-AVTP, when given to mice had no significant side effects but resulted in high levels of 6MP in the liver and gastrointestinal tract (S. Gunnarsdottir, A. A. Elfarra, Drug Metab. Dispos. (2003) Vol. 31, pp. 718-726). As shown in FIG. 1, cisAVTP was able to inhibit the in vitro replication of BVDV under conditions which did not affect overall cell viability.

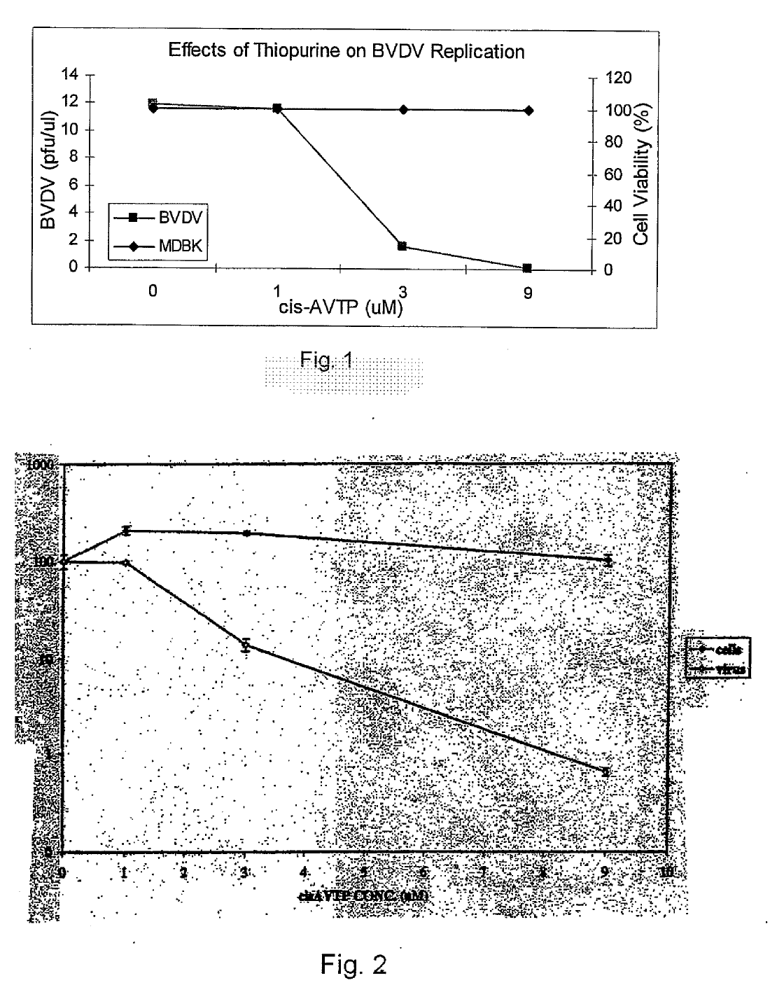

The compound cis-AVTP exhibits significant anti-viral activity without cellular cytoxicity at the concentrations assayed. FIG. 2 further illustrates that the antiviral effect of cis-AVTP is not dependent on cellular cytoxicity and occurs even when the route to cellular cytoxicity is blocked by the addition of 1 mM thymidine to the assayed cells. It can be observed that for increasing concentrations of cis-AVTP viral production was curtailed at concentrations when no decrease in cell growth was detected. When thymidine is present, DNA synthesis is limited, and there is therefore no significant incorporation of harmful building blocks into cellular DNA which would result in cytotoxicity. However, the viral suppression of cis-AVTP still occurs even though under conditions where no cellular toxicity is observed. Furthermore, this cellular state of high thymidine, low cell turnover may mimic physiological conditions (e.g. slow replicating liver tissue) better than rapidly dividing cells in a tissue culture dish, since few cells in situ are actively dividing at any one time.

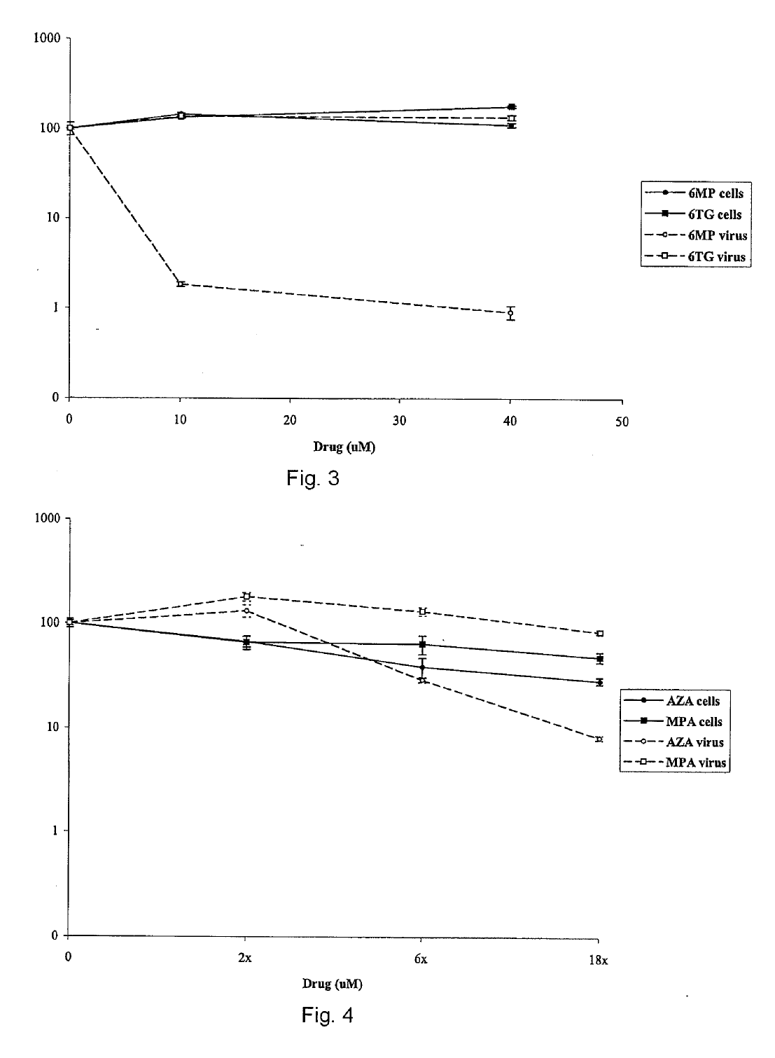

As noted above, metabolites of cis-AVTP were also assayed by the inventors for antiviral activity. The compounds 6-mercaptopurine, 6-methyl mercaptopurine and 6-thioguanine were individually added to MDBK cells with or without BVDV. 6-methyl mercaptopurine had no effect on cell growth or viral yield while both 6-mercaptopurine and 6-thioguanine both decreased cell growth and viral yield. To isolate the antiviral effect cells and virus were grown in the presence of 1 mM thymidine with either 6-mercaptopurine or 6-thioguanine. In the presence of 1 mM thymidine, 6-mercaptopurine caused a 2 fold log decrease in BVDV replication, while 6-thioguanine did not. These results are shown in FIG. 3 and demonstrate that certain metabolites of cis-AVTP exhibit antiviral activity while certain related compounds, also possessing cytotoxic properties, do not. In particular, 6-thioguanine, a compound possessing potent cytotoxic activity does not possess antiviral activity. 6-thioguanine is known to be an effective anti-cancer drug but its administration is accompanied by side-effects including bone marrow suppression with consequent anemia. In contrast, cis-AVTP is known to possess dramatically reduced systemic toxicity in large part due to its target specificity, discussed herein.

In anti-cancer applications, cis-AVTP is known to be a prodrug of 6-mercaptopurine whose activation is glutathione specific. In the anti-cancer setting, the administering of the prodrug, as opposed to the active agent (6-mercaptopurine) is desirable so as to avoid cytotoxicity issues associated with 6-mercaptopurine. This prodrug relationship therefore makes glutathione rich tissues targets of cis-AVTP as an anti-cancer agent. In this regard, hepatitis C virus (HCV) is the most common complication for liver transplantation in the developed countries. Indeed, recent medical reports indicate that mortality in liver transplant patients is on the increase. The presence of active HCV infection dramatically decreases patient survival and allograft survival in recipients of orthotropic liver transplantation. Assuming cis-AVTP's antiviral activity is due to a downstream metabolite, possibly 6-mercaptopurine, cis-AVTP's application as an antiviral agent is particularly attractive where the site of infection is glutathione rich tissue (e.g., liver, kidney and gastrointestinal tissues). As described herein, RNA viruses are inhibited by 6-mercaptopurine but this cis-AVTP metabolite, administered by itself, does not possess the tissue specificity of cis-AVTP and systemic toxicity is appreciable. Thus, an artisan will appreciate that cis-AVTP offers significant therapeutic advantage over prior compounds in terms of combined antiviral specificity and specificity of delivery.

While cis-AVTP will find its most likely application in tissues rich in glutathione, other applications of cis-AVTP may take advantage of targeting agents associated with the compound to direct the antiviral activity to a pre-selected tissue. Thusly, another potential application of cis-AVTP is in the treatment of RNA viral infection of the lung including RSV and severe acute respiratory syndrome (SARS). A model system for SARS is the murine hepatitis virus and cis-AVTP or virally-inhibiting derivatives, preferably having the structures of virally-inhibiting cis-AVTP metabolites, may be selectively activated in clinical therapy in the relevant lung tissues by, for example, chemical modification such that generation of 6-mercaptopurine is dependant on activation by lung specific metabolic enzymes such as those in the surfactant pathway. As well, optionally or in addition to chemical modifications, targeting agents such as monoclonal antibodies possessing lung specificity may be utilized to guide virally-inhibiting compounds to pre-selected tissues. Such targeting avoids the systemic toxicity issues associated with cis-AVTP metabolites. The above examples shall be understood to not be limiting as, the artisan reading the present disclosure will appreciate that cis-AVTP and derivatives thereof may be directed to a wide variety of pre-selected tissue targets using appropriate tissue-specific targeting agents, such targeting agents being known and available to the artisan.

Compound (I):

In view of the discoveries disclosed herein, the present invention utilizes a compound having the structure: 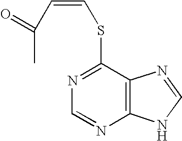
(compound I) having a pharmacological profile which makes it surprisingly effective and advantageous for anti-viral therapy while providing for much reduced side effects over previous agents. One particularly desirable advantage of compound (I) is reduced systemic toxicity and consequently reduced suppression of bone marrow in patients treated with compound (I) as compared to previous structurally-related agents.

The invention further includes methods utilizing derivatives of compound (I). The term “derivatives” includes but is not limited to compounds chemically- or biochemically-derived from compound (I) and possessing antiviral activity analogous thereto. Such derivatives preferably, but not necessarily, maintain the tissue specificity of the parent compound (I). All such derivatives will maintain analogous viral inhibiting characteristics as compound (I). Derivatives of compound (I) include ether derivatives, acid derivatives, amide derivatives, ester derivatives and the like, methods of manufacturing derivatives being widely-known in the pharmaceutical sciences. Derivatives also include isomers of compound (I). As defined herein, the term “isomer” includes, but is not limited to optical isomers and analogs, structural isomers and analogs, conformational isomers and analogs, and the like.

Additionally, this invention further includes methods of utilizing derivatives having the structure of known or yet to be determined compound (I) metabolites possessing antiviral activities analogous to compound (I). In general, the term “metabolite” includes any substance produced from another substance by metabolism or a metabolic process. For example, 6-mercaptopurine is understood to be a metabolite of cis-AVTP in glutathione rich tissues.

Preparation of Compound (I):

The compound cis-AVTP can be prepared following a synthesis route as described in Gunnarsdottir, et al., J. Pharmacol. Exp. Ther., 301: 77-86, 2002. The compounds employed as initial starting materials in the synthesis of cis-AVTP are known in the art, and to the extent not commercially available, are readily synthesized by standard procedures commonly employed in the art.

Assessment of Antiviral Activity of Compound (I):

The subject compound and compositions were demonstrated to have pharmacological activity in in vivo assays, e.g., they are capable of specifically modulating a cellular physiology to reduce an associated pathology or provide or enhance a prophylaxis. Certain preferred compositions are capable of specifically inhibiting or suppressing an RNA virus. The following assays are illustrative of methods by which the anti-viral activity of compound (I) may be demonstrated and assayed.

(A) Plaque Assays. Madin-Darby bovine kidney (MDBK) cells (ATCC CCL22) were grown in Dulbecco's modified Eagle's medium-F12 (Cellgro) supplemented with 10% heat-inactivated bovine serum (Atlanta Biologicals lot #k0041) that was demonstrated to be free of cytopathic and noncytopathic BVDV by ELISA and antibodies to BVDV type 1 strains by serum neutralization assay. The cells were also tested for BVDV contamination by reverse transcriptase (RT) PCR (15). Cytopathic, pNADL BVDV viral stock that had been extensively passed in this media were obtained.

Freshly seeded MDBK cell monolayers (1×10⁵ cells in a 100 mm dish) were seeded in the presence of varying concentrations of the compound and incubated for 3 hours at 37° C. Then a low multiplicity inoculum (0.01 pfu/cell of cp BVDV) was added and cells with virus were further incubated for 72 hours and the supernatant collected. Mock-infected plates with the same drug exposure for the same amount of time were trypsinized and counted in triplicate with a flow cytometer. Serial dilutions of each supernatant were added to freshly seeded monolayer without any drug. One hour after infection, the inoculum was removed and MDBK cell medium containing 1% methylcellulose was added to the monolayers. Plaques were counted 96 hours post-infection. Dilutions that gave approximately 25-75 plaques per plate were repeated in triplicate. FIG. 1 and 2 depict data demonstrating cis-AVTP's antiviral activity plus/minus the presence of 1 mM thymidine.

(B) Real time RT-PCR. HCV 1 bN replicon with no adaptive mutations was transfected into Huh7 cells (clone 1). (Ikeda, et al., J. Virol. 2002, 76:2997-3006.) Subconfluent cells were incubated for 72 hours in the presence of media containing 1 mM thymidine alone, or thymidine with 1 00 uM cis-AVTP. RNA was isolated with Trizol (Invitrogen) according to manufacturer's instructions, and 50 ng of total RNA was used per replicate of the real time PCR assay. Primers and probes for the HCV 5′ UTR as well as cellular GADPH were identical to those used by Cheney et al., Virology, 2002, 297:298-306. Samples were analyzed with the ABI 7700 Sequencer, and the ΔΔCt calculated as taught by Stuyver et al., Antimicrob. Agents Chem., 2003, 47: 244-54.

Moreover, as shown in FIG. 11, the riboside version of 6MP (6 thioinosine, 6TI in graph) has more antiviral properties than 6MP itself (2-3 fold). This is further evidence that the viral polymerase is the target of the drug. This also suggests that the antiviral properties is cis AVTP could be enhanced by making a riboside cis AVTP (or possibly trans AVTP riboside as discussed in Example II) since a nucleoside is closer to a substrate for the viral polymerase than a base

Compositions:

The present invention utilizes compositions which are suitable for pharmacological use. The term “viral-inhibiting effective amount” as used herein means an amount of a compound of formula (I) or a metabolite thereof which is capable of inhibiting RNA virus replication upon contact with the RNA virus. A “therapeutically effective amount” shall mean an amount of a compound of formula (I) or metabolite thereof which is capable of inhibiting RNA virus replication when administered to a host. A “host” shall include all animals susceptible to RNA virus infection, preferably a mammal and most preferably a human. The human may be a liver transplant patient inflicted with an HCV infection.

The RNA virus inhibition contemplated by the present method includes either therapeutic or prophylactic treatment, as appropriate. The specific dose of compound administered according to this invention to obtain therapeutic or prophylactic effects will, of course, be determined by the particular circumstances surrounding the case, including, for example, the compound administered, the route of administration, the condition being treated and the individual being treated. A typical daily dose will contain a dosage level of from about 0.01 mg/kg to about 50 mg/kg of body weight of an active compound useful in this invention. Preferred daily doses generally will be from about 0.05 mg/kg to about 20 mg/kg and ideally from about 0.1 mg/kg to about 10 mg/kg.

The compounds can be administered by a variety of routes including oral, rectal, subcutaneous, intravenous, intramuscular and intranasal. The compounds of the present invention are preferably formulated prior to administration. Therefore, the active ingredient in such formulation comprises from 0.01% to 99.9% by weight of the formulation.

By “pharmaceutically acceptable” it is meant that the carrier, diluent or excipient is compatible with the other ingredients of the formulation and not deleterious to the recipient thereof.

The present pharmaceutical formulations are prepared by known procedures using well-known and readily available ingredients. In making the compositions of the present invention, the active ingredient will usually be add mixed with a carrier or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus the compositions can be in the form of tablets, pills, powders, lozenges, sachets, cashettes, elixirs, suspensions, emulsions, solutions, syrups, aerosols, as a solid or in a liquid medium (ointments containing, for example, up to 10% by weight of the active compound, soft and hard gelatin capsules, suppositories, sterile injectable solutions, sterile package powders and the like. The term “active ingredient” means a compound according to formula (I) or a pharmaceutically acceptable salt thereof.

Hard gelatin capsules are prepared using the following ingredients: active ingredient (250 mg/capsule); starch, dried (200 mg/capsule); magnesium stearate 10 mg/capsule); total 460 mg/capsule).

Methods of Use:

The present invention provides novel methods for the use of the foregoing compound (I) and metabolites thereof possessing antiviral activity in antiviral compositions. In particular, the invention provides novel methods for treating or preventing RNA viruses, preferably from the flavivirus family. The methods typically involve administering to a patient an effective formulation of one or more of the subject compositions.

The invention provides methods of using the above-described compound (I) and compositions containing the same to treat RNA virus-related infections or diseases or provide medicinal prophylaxis to individuals who are in need thereof. These methods generally involve administering to the host an effective amount of the subject compounds or pharmaceutically acceptable compositions.

The compounds and compositions may be advantageously combined and/or used in combination with other anti-viral agents useful in the treatment and/or prevention of the viral infections described herein. Suitable agents for combination therapy include those that are currently commercially available and those that are in development or will be developed. The compositions and compounds of the invention and the pharmaceutically acceptable salts thereof can be administered in any effective way and further examples describing such are found in the Examples section below.

### EXAMPLE II

Trans-AVTP as an Antiviral Agent:

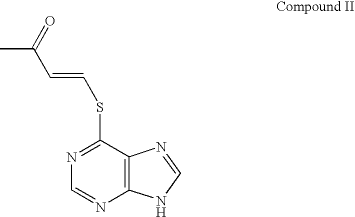

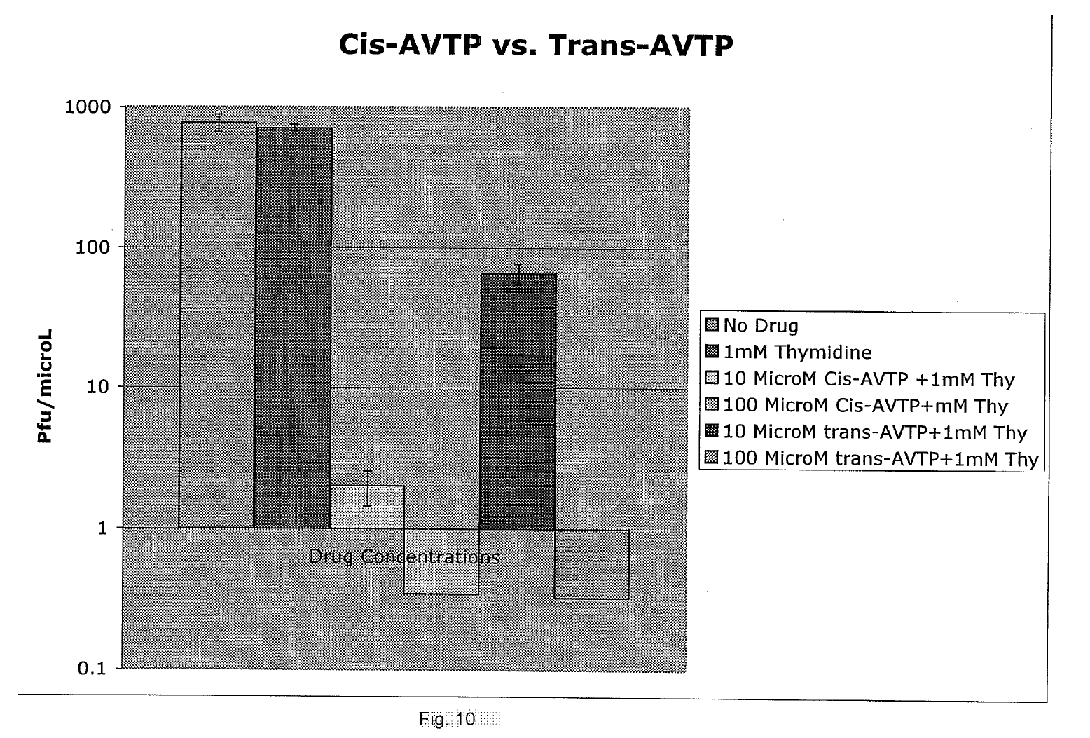

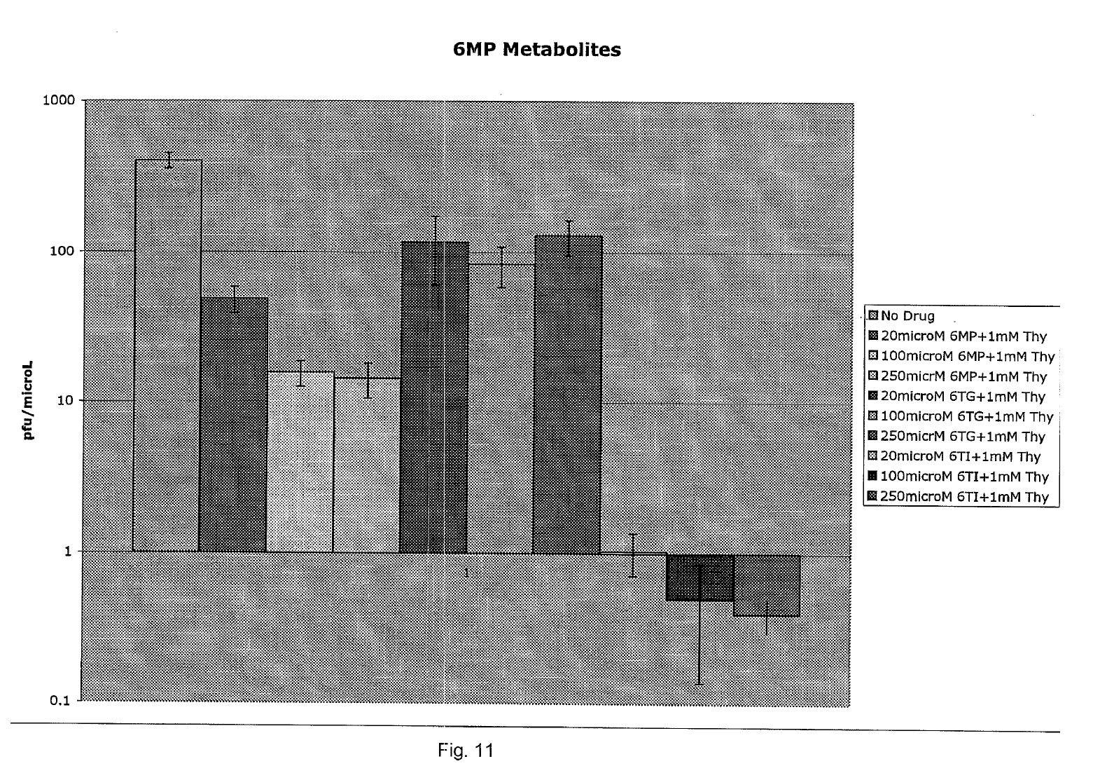

FIG. 10 and the table below demonstrate that an isomer of cis AVTP (trans AVTP) also has antiviral properties. Trans-AVTP is represented by compound II.

| | | | 10 microM | 100 microM | 10 microM | 100 microM |
|---|---|---|---|---|---|---|
| | No Drug | Thymidine | cis | cis | trans | trans |
| Pfu/microL | 790 | 670 | 1.4 | 0.34 | 53 | 0.32 |
| | 660 | 740 | 2.5 | 0.35 | 73 | 0.38 |
| | 880 | 730 | 2.1 | 0.36 | 71 | 0.29 |
| Avg | 776.666667 | 713.333333 | 2 | 0.35 | 65.66666667 | 0.33 |
| St. dev | 110.6044 | 37.859389 | 0.556776436 | 0.01 | 11.01514109 | 0.045825757 |

As shown in the table above, at 10 uM trans-AVTP has less antiviral than cis-AVTP, however, both compounds demonstrate similar levels of antiviral activity at 100 uM. Accordingly, administering both compound I and II may lead to even higher liver levels of the active agent. Trans-AVTP may therefore be useful when used by itself or when used in combination with cis-AVTP. While not adopting any one mechanism of action herein, this may be based on the fact that the trans-AVTP compound may act to decrease or decoy elimination of cis-AVTP in a whole animal model.

As described above in example I, trans-AVTP like cis-AVTP may be synthesized, characterized and assessed by methods known to one of ordinary skill in the art. Derivatives of the compounds may also be prepared, as shown in example I. Similarly, trans-AVTP will also have pharmaceutically acceptable carrier and methods of use as discussed for cis-AVTP. Further, like cis-AVTP, trans-AVTP will find its preferred application in tissues rich in glutathione, such as liver, kidney and GI tract.

### EXAMPLE II

Effect of Antimetabolite Immunosuppressants on Flaviviridae Including Hepatitis C Virus

Background: Reoccurrence of Hepatitis C Virus (HCV) after liver transplantation is almost universal, and decreases both graft and patient survival. Medications that alter nucleic acid metabolism, including some common immunosuppressants used in HCV infected patients, may affect viral replication.

Methods: Bovine Viral Diarrhea Virus (BVDV) is in the Flaviviridae family and closely related to HCV. The inventors measured the effect of two immunosuppressants, azathioprine (AZA) and mycophenolate (MPA) on both BVDV replication by plaque assay, and host cell replication by flow cytometry. The inventors also compared the effect of ribavirin and AZA on the level of HCV replicon RNA by real time RT-PCR.

Results: At doses that achieved similar cytoxicity, AZA decreased BVDV replication 10 fold more than MPA. The inhibition of BVDV by AZA occurred at lower doses than the cellular cytotoxicity and does not depend on cell cytotoxicity. A two log reduction in viral titers occurred without cytoxicity of AZA was blocked by inhibiting ribonucleotide reductase with high concentrations of thymidine. A metabolite of AZA, 6-mercaptopurine, still possessed this antiviral effect, but a metabolite further downstream, 6-thioguanine, did not, even though 6-thioguanine is the metabolite responsible for cellular toxicity. The effect of AZA on a HCV replicon was at least as large as that of ribavirin.

Hepatitis C Virus (HCV) is the most common indication for liver transplantation in developed countries. Reinfection of liver allografts by HCV is virtually universal. Moreover, the natural history of HCV is accelerated post-liver transplantation and there are reports that mortality post liver transplant for HCV is actually increasing recently. The presence of active HCV infection decreases patient survival and liver allograft survival in receipents of orthotopic liver transplantation. HC viremia usually exceeds pretransplantation levels, and it is unknown whether new specific viral variants are selected after transplantation. Additionally the amount each individual immunosuppressant drug contributes to the acceleration of the natural history of HCV is controversial. Answering these questions is difficult in the absence of cell culture models for HCV.

Ribavirin has been shown to enhance the activity of interferon in the treatment of HCV and increase the percentage of sustained responders. The exact mechanism of this enhancement is unknown, though at high concentrations ribavirin can be incorporated into RNA viral genomes and decrease replication. Antimetabolite immunosuppressants have some commonalities with ribavirin. Both ribavirin and mycophenolate (MPA) inhibit inosine monophosphate dehydrogenase. This and other data has led some to quantify the antiviral affect of mycophenolate and other inosine monophosphate dehydrogenase inhibitors. At the same time ribavirin and 3 metabolites of azathioprine (AZA) are all processed to monophosphate nucleotides by inosine monophosphate dehydrogenase intracellularly which then compete with endogenous nucleotide pools. Controlled clinical trials investigating potential antiviral effects of either AZA or MPA, and which immunosuppressive cocktail is associated with the least HCV reoccurrence are conflicting. These trials are also difficult to compare given the number of variables such as genotypically different viruses, different immunosuppressive cocktail, and different patient populations. To address the limited question of whether MPA or AZA have specific antiviral effects because of their similarities to ribavirin, the inventors turned to a virus in the same viral family and closely related to HCV, Bovine Viral Diarrhea Virus (BVDV), which can be grown in cell culture. Using BVDV as a surrogate of HCV the inventors directly measured the antiviral effects of antimetabolite immunosuppressants independent of their effects on the adaptive immune system. In this embodiment the inventors demonstrated significantly more specific antiviral activity of AZA than MPA. Additionally the inventors show that the antiviral activity of AZA is comparable to that of ribavirin itself on a HCV replicon.

Materials and Methods: Madin-Darby bovine kidney (MDBK) cells (ATCC CCL22) were grown in Dulbecco's modified Eagle's medium-F12 (Cellgro) supplemented with 10% heat-inactivated bovine serum (Atlanta Biologicals lot #k0041) that was demonstrated to be free of cytopathic and noncytopathic BVDV by ELISA and antibodies to BVDV type 1 strains by serum neutralization assay. The cells were also tested for BVDV contamination by reverse transcriptase (RT) PCR. Cytopathic, pNADL BVDV viral stock that had been extensively passed in this media was kindly provided by Ron Schultz, University of Wisconsin. AZA, MPA, 6-methyl mercaptopurine, 6-thioguanine and thymidine were purchased from Sigma. 6-mercaptopurine and ribavirin were purchased from ICN.

Plaque assays: Generally, freshly seeded MDBK cell monolayers (1×10⁵ cells in a 100 mm dish) were seeded in the presence of varying concentrations of antimetabolite drugs and incubated for 3 hours at 37° C., 5°CO₂. Then a low multiplicity inoculum (˜0.01 pfu/cell of cp BVDV) was added and cells with virus were further incubated for 72 hrs after which the supernatant was collected. Mock-infected plates with the same drug exposure for the same amount of time were trypsinized and counted in triplicate with a flow cytometer. Serial dilutions of each supernatant were added to 4 hr old, newly seeded monolayer without any drug. One hour after infection, the inoculum was removed and MDBK cell medium containing 1% methylcellulose was added to the monolayers. Plaques were counted 96 h postinfection. Dilutions that gave ˜25-75 plaques per plate were repeated in triplicate.

Real time RT-PCR: HCV 1 bN replicon with no adaptive mutations transfected into Huh7 cells (clone 1) was kindly supplied by Stanley Lemon, University of Texas, Galveston. Subconfluent cells were incubated for 72 hours in the presence of media containing 1 mM thymidine alone, or thymidine with either 100 uM Ribavirin or 100 uM AZA. RNA was isolated with Trizol (Invitrogen) according to manufacturers instructions, and 50 ng of total RNA was used per replicate of the real time PCR assay. Primers and probes for the HCV 5′ untranslated region as well as cellular glyceraldehydes-3-phosphate dehydrogenase) were identical to that used by Cheney et al. Samples were analyzed with the ABI 7700 Sequencer, and the ΔΔCt calculated according to Stuyver.

Results: Comparison of azathioprine and mycophenolate on Bovine Viral Diarrhea Virus To begin to look for selective pressure that altered nucleotide pools may place on RNA viruses the inventors measured BVDV growth in Martin Darby Bovine Kidney (MDBK) cells exposed to MPA or AZA. When cells were actively replicating (subconfluent cells), both MPA and to a much lesser extent AZA had cytostatic/toxic effects in addition to any potential specific antiviral effects (data not shown). The cytostatic/toxic effects of MPA, presumably attributable to a decreased de novo purine synthesis from IMPDH inhibition, were such that even very low concentrations of MPA killed all the cells when the cells were rapidly dividing. By allowing cells to reach confluency and a slower growth rate before exposing cells to drug and virus though, the effect of the drug on viral replication on an intact cell monolayer could be evaluated (FIG. 4). Under these conditions the antiviral effect of AZA was larger than that of MPA, with only 12% of the virus produced per living cell grown in AZA, compared to the amount of virus produced per living cell grown in MPA when concentrations of both drugs caused an approximately similar (˜50%) decrease in cell growth.

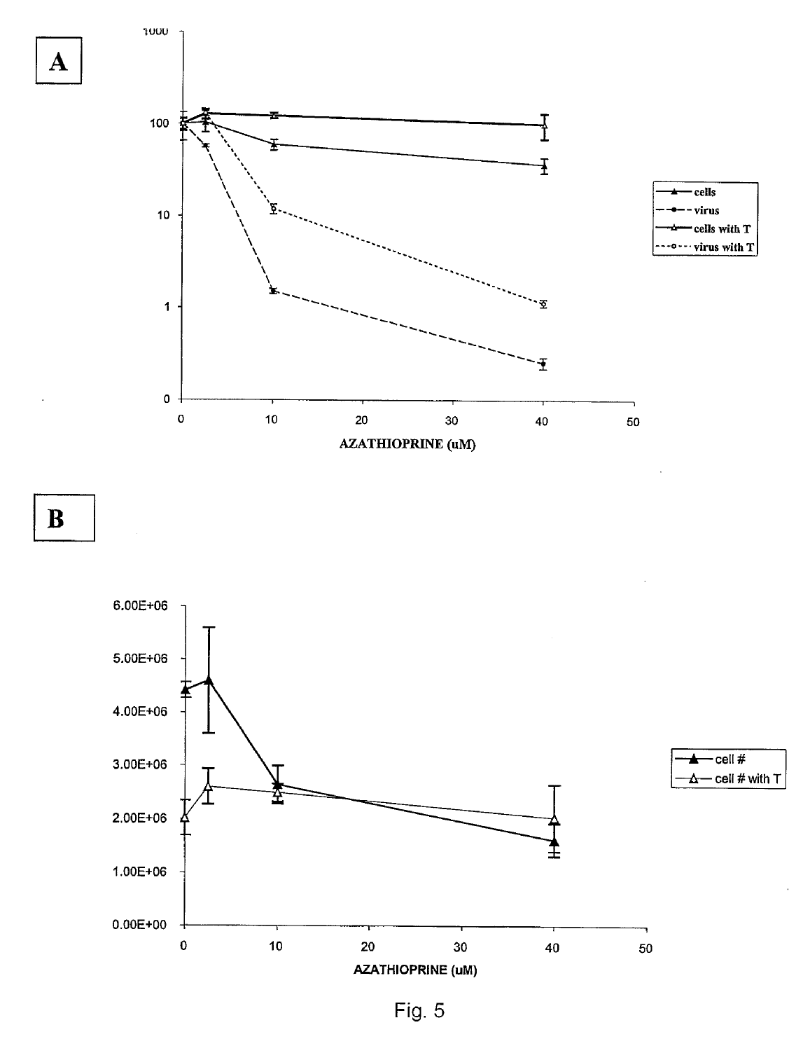

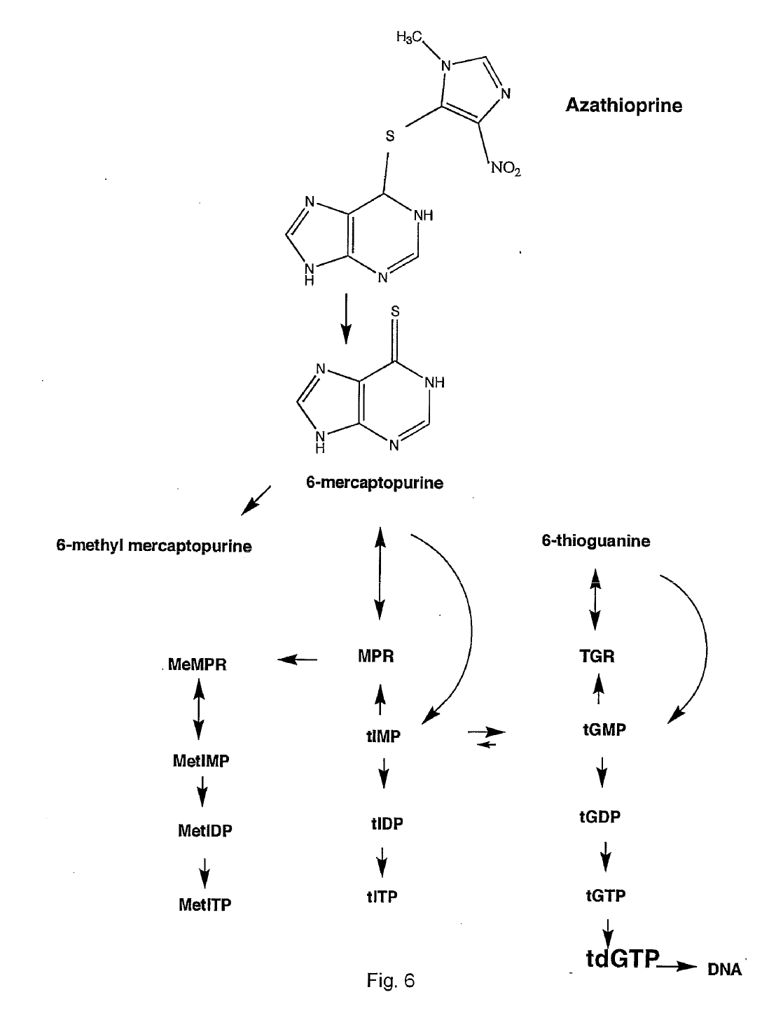

The effect of azathioprine on Bovine Viral Diarrhea Virus does not depend on cytotoxic effects. The cytostatic/toxic effects of AZA are more modest than MPA and thus allowed the addition of AZA to rapidly dividing cells, which in turn allowed more robust viral production. With increasing concentrations of AZA, viral production was significantly curtailed, even at concentrations when no detectable decrease in cell growth occurred (FIG. 5). The decrease in cell growth caused by AZA at higher concentrations can be prevented by high concentrations of thymidine. Under these conditions DNA synthesis was limited, so there should be no production and incorporation of the metabolite of AZA, 6-thioguanosine triphosphate, into cellular DNA and FIG. 6, and therefore no cytotoxicity. The viral suppression (FIG. 5) still occurred even though under these conditions AZA caused no change in host cell growth. Since only a small minority of hepatocytes in a diseased liver, (and even smaller minority in a normal liver) are actively dividing, this cellular state of high thymidine, low cell turnover may mimic liver tissue better than rapidly dividing cells in a tissue culture dish. The number of BVDV plaques produced was decreased by slowing cellular growth either by cells reaching confluency (data not shown, similar to the behavior of the HCV replicon), or by thymidine (compare FIG. 5b, 0 uM AZA with and without thymidine) and demonstrates the effect of the cellular milieu on viral replication rates.

6-mercaptopurine, but not the downstream metabolite, 6-thioguanine, is the likely mediator of azathioprine's antiviral effect. To begin to determine the mechanism of the antiviral effect of AZA, the inventors examined whether any of the metabolites of AZA (FIG. 6) also had an antiviral effect. 6-methyl mercaptopurine, 6-mercaptopurine and 6-thioguanine were each individually added to MDBK cells with or without BVDV. 6-methyl mercaptopurine had no effect on cell growth or viral yield while both 6-mercaptopurine and 6-thioguanine decreased cell growth and viral yield (data not shown). To isolate the antiviral effect, cells and virus were grown in the presence of thymidine with either 6-mercaptopurine or 6-thioguanine (FIG. 3). In the presence of thymidine neither azathioprine metabolite had an appreciable effect on cell growth consistent with the prodrug AZA itself, but 6-mercaptopurine still caused a 2 log decrease in BVDV replication, while 6-thioguanine did not.

HCV replicon is more sensitive to azathioprine than ribavirin at equivalent doses. Cell confluency affects the amount of HCV replicon per cell, and since high concentrations of AZA without thymidine does affect Huh7 cell confluency (data not shown), the inventors also tested the HCV replicon in the presence of thymidine. Huh7 cells bearing the 1 bN replicon were grown in the presence of 1 mM thymidine with no other drug, AZA, or ribavirin. After 72 hours, total RNA was isolated and equivalent amounts assayed by Real time RT-PCR with probes to the viral 5′ viral untranslated region and a cellular housekeeping gene to normalize the results. AZA reliably produced almost a 1 cycle increase in the number of cycles required to teach the critical threshold (Cₜ) compared to no drug, which corresponds to only ˜50% as much replicon in cells exposed to AZA (FIG. 7). While this change was small compared to the effect of AZA on BVDV it was of similar magnitude or larger than the change on the HCV replicon due to equimolar amounts of ribavirin.

Discussion: Azathioprine has been used in liver transplantation for more than 30 years. It is a prodrug that is converted to 6-mercaptopurine and eventually into 6-thioinosine and 6-thioguanosine triphosphate (FIG. 6). The triphosphate of 6-thioguanosine is converted to deoxy6-thioguanosine and incorporated into cellular DNA. The thioribonucleotides though are available for inhibition and/or incorporation by viral enzymes including the RNA polymerase. Therefore AZA could share some proposed mechanisms with ribavirin that are dependent upon both drugs being triphosphorylated and recognized by the viral replication machinery. The effect of ribavirin monotherapy, or any anti HIV nucleoside monotheray is quite small, and at least in the case of HIV nucleosides quickly obscured by the selection of viral resistance. In certain situations the selection of less fit HIV viruses with mutant polymerases can be clinically preferable than wild type virulent HIV. A similar detailed understanding how to select mutant HCV viruses is lacking even though the HCV polymerase is the likely target of ribavirin and possibly an indirect target of other antimetabolite drugs including inosine monophosphate dehydrogenase inhibitors. Ribavirin monophosphate is a competitive inhibitor of inosine monophosphate dehydrogenase and is further phosphorylated to a triphosphate nucleotide analog, which at least in vitro is a substrate for the HCV genotype 1b polymerase. Metabolites of azathioprine also decrease purine synthesis through a different mechanism (inhibition of glutamine-5-phosphoribosylpyrophosphate amidotransferase), while MPA and ribavirin both inhibit GTP synthesis by inhibiting inosine monophosphate dehydrogenase. Since only 6-mercaptopurine has an antiviral effect and not 6-thioguanine, but both 6-mercaptopurine and 6-thioguanine decrease purine synthesis, the indirect effects of AZA on purine synthesis do not seem to be sufficient for the antiviral activity. These results are consistent with two recent studies that show ribavirin has weak antiviral (Hepatitis GB) or anti-HCV replicon activity, but MPA has none. This is the first study to demonstrate antiviral activity of AZA, or compare the magnitude to MPA and ribavirin.

Mycophenolate mofetil, the prodrug of MPA clearly decreases risk of rejection relative to AZA in clinical trials. Yet even in the face of more rejection AZA has been associated with a variable amount of HCV reoccurrence. One study showed less reoccurrence with an AZA containing regimen versus a non AZA containing regimen, while another found more HCV reoccurrence in patients with higher doses of AZA and corticosteriods. Meanwhile mycophenolate mofetil has also been associated with less HCV reoccurrence than AZA, no benefit compare to a regimen without mycophenolate mofetil or AZA, or an increased risk of graft failure.

In this example, the inventors demonstrate that azathioprine causes a specific antiviral effect independent of its effect on adaptive immunity. This effect is larger than that of mycophenolate and maybe more closely related to the mechanism of ribavirin. The inventors present evidence that the effect is mediated by a thioinosine metabolite perhaps by being incorporated as has been suggested to occur with ribavirin. If it is incorporated then the antiviral effect could be mediated either through the induction of mutations, or by altering RNA structure, which in turn may effect enzyme processivity, ribosome translation or other properties of an RNA genome. The antiviral effect of AZA is at least as large as the antiviral effect of ribavirin on a HCV1 bN replicon. With this effect in mind the role of azathioprine in liver transplantation should be reevaluated to determine if AZA causes a transient decrease in viral load or selection of AZA resistant HCV occurs.

### EXAMPLE IV

Purine Nucleoside Analogs as Broad Spectrum Antivirals for Emerging RNA Viral Pathogens

Present embodiment investigates and teaches the use of the thiopurine class of nucleoside analogs as a broad spectrum antiviral for use in positive RNA viral infections.

Thiopurine nucleoside analog compounds have broad spectrum anti-RNA virus activities, and will be effective at inhibiting the replication of multiple related and unrelated viruses. Generally, inventors teach effectiveness of antiviral compounds in tissue culture system and effectiveness of antiviral compounds in cell free polymerase assay. The effectiveness study for the tissue culture system may be done by determining if a compound inhibits viral replication, is selective for viral resistance or if a drug induced mutations are linked to resistance. The effectiveness study for polymerase assay may be done by determining if a viral polymerase incorporates analog compounds during RNA transcription and if that analog incorporation affects translation and protein function of transcribed RNA.

Effectiveness of Antiviral Compounds in Tissue Culture System.

To examine antiviral activity of thiopurines the inventors will infect susceptible cells with different RNA viruses and determine their relative effects. In order to test the broad spectrum potential of these compounds viruses from two distinct positive sense RNA viral families may be used. Bovine viral diarrhea virus (BVDV) and the yellow fever virus (YFV) are representative members of the flavivirus family and Infectious bronchitis virus (IBV), transmissible gastroenteritis virus (TGEV), and bovine coronavirus (BCoV) represent the three known antigenic groups of the coronavirus family. All of these viruses may be propagated in African green monkey kidney (Vero) cells, except BVDV which will be propagated in Madin-Darby bovine kidney (MDBK) cells. To determine the affects of thiopurine analogs on the replication of these viruses, they will be propagated in the presence of nucleoside analogs 6-mercaptopurine (6MP), cis-6-(2-acetylvinylthio) purine (cis-AVTP). Testing for their potential broad spectrum activities and comparative effects to that of ribavirin the only approved broad spectrum antiviral for RNA virus infections may be consequently studied.

Dilutions of the antiviral compounds will be titrated in each respective cell line to determine maximum nontoxic dose using a trypan blue exclusion viability assay. Effectiveness of each of these compounds to inhibit flavivirus and/or coronavirus replication will be determined by assaying for reduced viral titers. Changes in titer will be measured by standard tissue culture infectious dose₅₀ (TCID₅₀) assays and viral plaque assays.

To determine how these analogs exact their antiviral effects on each of the above viruses, drug resistant mutants will be selected. Each virus will be propagated in the presence of each inhibiting compound at a concentration one third the viricidal dose. Viruses will be passaged five times in the presence of antiviral compound, and then tested for development of drug resistance at the inhibiting concentration. Virus isolates capable of replicating in the presence of antiviral compound will then be plaque purified and amplified. Mutants will be compared to parental virus by plaque morphology and in differences in growth rates using one step growth curve assays.

The sequence of drug resistant isolates will be compared to the sequence of control viruses propagated at the same time, with the same passage history, in the absence of drug, and analyzed for mutations which may explain their resistance. Previous studies with drug resistant RNA viruses suggest that mutations in the polymerase gene can affect the polymerase recognition of these analog compounds. Therefore the experiments will first examine the polymerase and replicase regions of these viruses for possible mutations, however if no mutations are observed other areas of each genome will be examined. To ensure that any mutation detected is a result of drug selection and not a spontaneous mutation, selection will be performed a minimum of three times, and after each selection a minimum of three drug resistant isolates will be plaque purified. Mutations consistently found in independently generated isolates will be considered involved in conferring drug resistance. Additionally, these mutations will then be introduced into the parent virus using infectious clone and reverse genetic techniques. These in vitro generated mutants will then be tested a drug resistant phenotype.

All three compounds are expected to inhibit replication of flavivruses. Studies in the inventors' laboratory and others have previously demonstrated the in vitro effectiveness of these compounds to inhibit members of the flavivirus family. It is also expected that the thiopurine compounds 6MP and cis-AVTP will inhibit the in vitro replication of coronaviruses. However, previous in vivo studies with ribavirin and coronavirus infections suggest that ribavirin will not reduce the in vitro replication of IBV, TGEV, or BCoV. Finally, will be able to determine and select drug resistant mutants for each of the active compounds.

Effectiveness of Antiviral Compounds in Cell Free Polymerase Assay.

To determine the ability of viral polymerases from flaviviruses and coronaviruses to incorporate these nucleoside analogs, the polymerase genes from BVDV and IBV will be cloned, expressed in bacteria as histidine fusion protein, and affinity purified. If dramatic differences are seen with a viral family, these polymerases may also be used. Its ability to transcribe RNA from a template will then be tested in a RNA primer extension assay (FIG. 1). The sequence of the copied RNA template will then be analyzed utilizing mass spectrometry techniques. The error rate of each normal viral polymerase with normal nucleosides can be calculated. The inventors will then repeat these experiments in the presences of nucleoside analogs. The incorporation rates of these analogs per RNA molecule generated will be determined by mass spectrometry. Additionally, if mutations correspond to the viral polymerase, then these mutant polymerases will be tested in the primer extension assay to determine if the mutation affects the rate with which analogs are incorporated.

To determine if incorporation of nucleoside analogs in transcribed RNA has a functional effect primer extension assay will be performed as described above, however the template RNA will be the complement strand of a messenger RNA encoding a luciferase reporter gene under the control of an internal ribosome entry site (IRES). In vitro transcribed mRNA will be translated under cell free conditions and the amount of luciferase bio-elumination measured using standard protocols.

It is expected that the recombinant polymerase proteins to incorporate the antiviral compounds which had antiviral effects on their respective whole virus. This incorportation rate will translate in an increase in the polymerase error rate, and will have a mutagenizing effect on the template RNA. Further experiments using the luciferase reporter construct will demonstrate that RNAs generated in the presences of these effective compounds will have a diminished capacity to produce functional luciferase protein as determined by decreased bio-lumination

Preliminary Data has established that BVDV replication is inhibited by thiopurines. It has been demonstrated that flaviviruses are sensitive to ribavirin, however the exact mechanism of action for this inhibition is unclear. It is also unclear why ribavirin works for some RNA viruses and not others. In order to understand how these compounds work the inventors studied other nucleoside analogs that will inhibit flavivirus replication. The inventors have previously demonstrated that the flavivirus BVDV is sensitive thiopurine compounds AZA, 6MP, and cis-AVTP (FIG. 1). Using the compounds 6MP and AZA the inventors were able to demonstrate a significant reduction in the out put of infectious BVDV as compared with virus propagated without either of these compounds.

Generation of thiopurine resistant BVDV. The inventors have also selected for BVDV mutants which are resistance to thiopurines (FIG. 8). BVDV was propagated in the presence of AZA for certain number of passages. The virus was then harvested and plaque assays performed without AZA. The results were a BVDV mutant which is resistant to AZA (FIG. 10B). Preliminary studies underway for isolating and characterizing the mutations in the drug resistant BVDV suggests that mutations in the viral polymerase are involved in drug resistance.

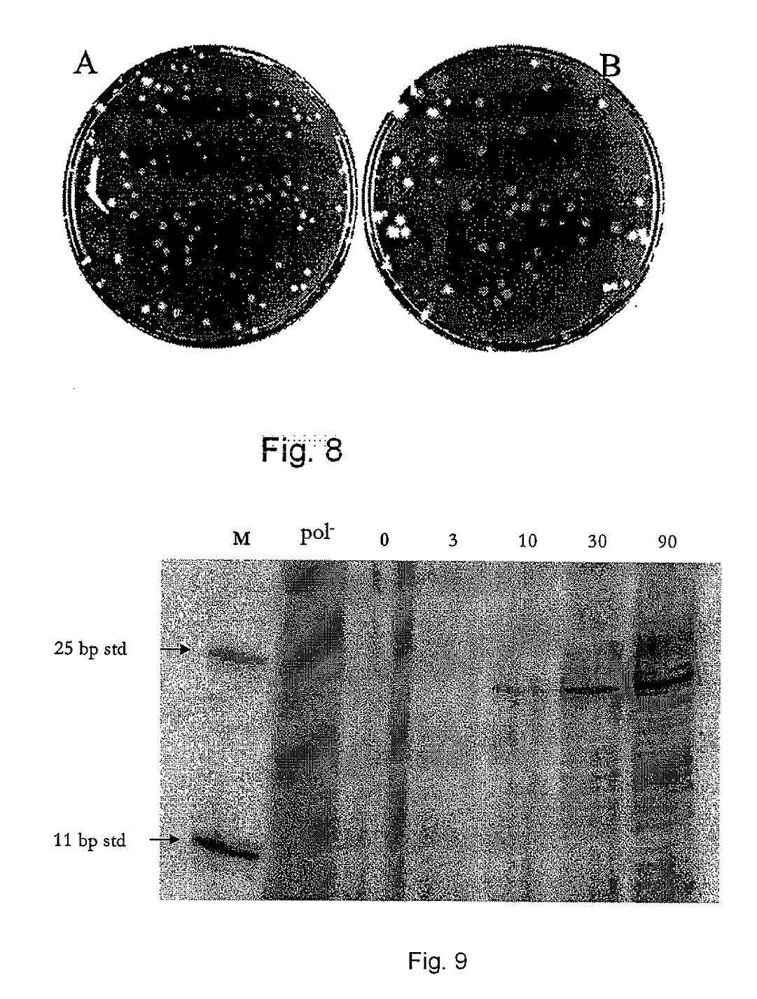

Recombinant RNA-dependant RNA polymerase transcribes RNA templates in vitro. To understand how these compounds affect viral replication, experiments were conducted to measure the relative rates at which the viral polymerases will incorporate these compounds in growing strands of RNA. Recently developed in vitro assay for measuring polymerase function and determining function may be useful in this respect. The polymerase of gene of the flavivirus HCV has been expressed in bacteria cells and purified. Since this protein has demonstrated enzymatic activity, it will transcribe RNA from a RNA template in a cell free assay, and further, the transcribed RNAs may be detected as shown in FIG. 9.

Those skilled in the art will recognize, or be able to ascertain using no more then routine experimentation, numerous equivalents to the specific methods, assays and reagents described herein. Such equivalents are considered to be within the scope of this invention and covered by the following claims. All publications, patents, and patent applications cited herein are hereby incorporated by reference in their entirety for all purposes.

### III. REFERENCES

(1)Berenguer, M., Natural history of recurrent hepatitis C. Liver Transpl, 2002. 8(10 Suppl 1): p. S14-8.
(2)Forman, L. M., J. D. Lewis, J. A. Berlin, H. I. Feldman, and M. R. Lucey, The association between hepatitis C infection and survival after orthotopic liver transplantation. Gastroenterology, 2002. 122(4): p. 889-96.
(3)Gane, E. J., N. V. Naoumov, K. P. Qian, M. U. Mondelli, G. Maertens, B. C. Portmann, J. Y. Lau, and R. Williams, A longitudinal analysis of hepatitis C virus replication following liver transplantation. Gastroenterology, 1996. 110(1): p. 167-77.
(4)Everson, G. T., Impact of immunosuppressive therapy on recurrence of hepatitis C. Liver Transpl, 2002. 8(10 Suppl 1): p. S19-27.
(5)McHutchison, J. G., S. C. Gordon, E. R. Schiff, M. L. Shiffman, W. M. Lee, V. K. Rustgi, Z. D. Goodman, M. H. Ling, S. Cort, and J. K. Albrecht, Interferon alfa-2b alone or in combination with ribavirin as initial treatment for chronic hepatitis C. Hepatitis Interventional Therapy Group. N Engl J Med, 1998. 339(21): p. 1485-92.
(6)Crotty, S., D. Maag, J. J. Arnold, W. Zhong, J. Y. Lau, Z. Hong, R. Andino, and C. E. Cameron, The broad-spectrum antiviral ribonucleoside ribavirin is an RNA virus mutagen. Nat Med, 2000. 6(12): p.1375-9.
(7)Willis, R. C., D. A. Carson, and J. E. Seegmiller, Adenosine kinase initiates the major route of ribavirin activation in a cultured human cell line. Proc Natl Acad Sci USA, 1978. 75(7): p. 3042-4.
(8)Markland, W., T. J. McQuaid, J. Jain, and A. D. Kwong, Broad-spectrum antiviral activity of the IMP dehydrogenase inhibitor VX-497: a comparison with ribavirin and demonstration of antiviral additivity with alpha interferon. Antimicrob Agents Chemother, 2000. 44(4): p. 859-66.
(9)Nelson, J. A., J. W. Carpenter, L. M. Rose, and D. J. Adamson, Mechanisms of action of 6-thioguanine, 6-mercaptopurine, and 8-azaguanine. Cancer Res, 1975. 35(10): p. 2872-8.
(10) Wiesner, R., et al., A randomized double-blind comparative study of mycophenolate mofetil and azathioprine in combination with cyclosporine and corticosteroids in primary liver transplant recipients. Liver Transpl, 2001. 7(5): p. 442-50.
(11) Jain, A., R. Kashyap, A. J. Demetris, B. Eghstesad, R. Pokharna, and J. J. Fung, A prospective randomized trial of mycophenolate mofetil in liver transplant recipients with hepatitis C. Liver Transpl, 2002. 8(1): p. 40-6.
(12) Hunt, J., F. D. Gordon, W. D. Lewis, E. Pomfret, J. J. Pomposelli, R. L. Jenkins, and U. Khettry, Histological recurrence and progression of hepatitis C after orthotopic liver transplantation: influence of immunosuppressive regimens. Liver Transpl, 2001. 7(12): p.1056-63.
(13) Berenguer, M., M. Prieto, J. Cordoba, J. M. Rayon, D. Carrasco, V. Olaso, F. San-Juan, M. Gobernado, J. Mir, and J. Berenguer, Early development of chronic active hepatitis in recurrent hepatitis C virus infection after liver transplantation: association with treatment of rejection. J Hepatol, 1998. 28(5): p. 756-63.
(14) Burak, K. W., W. K. Kremers, K. P. Batts, R. H. Wiesner, C. B. Rosen, R. R. Razonable, C. V. Paya, and M. R. Chariton, Impact of cytomegalovirus infection, year of transplantation, and donor age on outcomes after liver transplantation for hepatitis C. Liver Transpl, 2002. 8(4): p. 362-9.
(15) Becher, P., M. Orlich, M. Konig, and H. J. Thiel, Nonhomologous RNA recombination in bovine viral diarrhea virus: molecular characterization of a variety of subgenomic RNAs isolated during an outbreak of fatal mucosal disease. J Virol, 1999. 73(7): p. 5646-53.
(16) Ikeda, M., M. Yi, K. Li, and S. M. Lemon, Selectable subgenomic and genome-length dicistronic RNAs derived from an infectious molecular clone of the HCV-N strain of hepatitis C virus replicate efficiently in cultured Huh7 cells. J Virol, 2002. 76(6): p. 2997-3006.
(17) Cheney, I. W., S. Naim, V. C. Lai, S. Dempsey, D. Bellows, M. P. Walker, J. H. Shim, N. Horscroft, Z. Hong, and W. Zhong, Mutations in NS5B Polymerase of Hepatitis C Virus: Impacts on in Vitro Enzymatic Activity and Viral RNA Replication in the Subgenomic Replicon Cell Culture. Virology, 2002. 297(2): p. 298-306.
(18) Stuyver, L. J., et al., Ribonucleoside analogue that blocks replication of bovine viral diarrhea and hepatitis C viruses in culture. Antimicrob Agents Chemother, 2003. 47(1): p. 244-54.
(19) Negro, F., M. Papotti, S. Taraglio, L. Rubbia-Brandt, E. Giostra, D. Pacchioni, M. Rizzetto, and A. Hadengue, Relationship between hepatocyte proliferation and hepatitis delta virus replication in neoplastic and non-neoplastic liver tissues. J Viral Hepat, 1997. 4(2): p. 93-8.
(20) Guo, J. T., V. V. Bichko, and C. Seeger, Effect of alpha interferon on the hepatitis C virus replicon. J Virol, 2001. 75(18): p. 8516-23.
(21) Van Scoik, K. G., C. A. Johnson, and W. R. Porter, The pharmacology and metabolism of the thiopurine drugs 6-mercaptopurine and azathioprine. Drug Metab Rev, 1985. 16(1-2): p. 157-74.
(22) Loveday, C., International perspectives on antiretroviral resistance. Nucleoside reverse transcriptase inhibitor resistance. J Acquir Immune Defic Syndr, 2001. 26 Suppl 1: p. S10-24.
(23) Maag, D., C. Castro, Z. Hong, and C. E. Cameron, Hepatitis C virus RNA-dependent dependent RNA polymerase (NS5B) as a mediator of the antiviral activity of ribavirin. J Biol Chem, 2001. 276(49): p. 46094-8.
(24) Lanford, R. E., D. Chavez, B. Guerra, J. Y. Lau, Z. Hong, K. M. Brasky, and B. Beames, Ribavirin induces error-prone replication of GB virus B in primary tamarin hepatocytes. J Virol, 2001. 75(17): p. 8074-81.
(25) Zhou, S., R. Liu, B. M. Baroudy, B. A. Malcolm, and G. R. Reyes, The effect of ribavirin and IMPDH inhibitors on hepatitis C virus subgenomic replicon RNA. Virology, 2003. 310(2): p. 333-42.
(26) Gunnarsdottir, et al., Novel Glutathione-dependent Thiopurine Prodrugs: Evidence for Enhanced Cytotoxicity in Tumor Cells and for Decreased Bone Marrow Toxicity in Mice. J. Pharmal. Exp. Therapeutics. 2002. 301(1): p. 77-86.
(27) Gunnarsdottir, et al., The Glutathione-activated Thiopurine Prodrugs trans-6-(2-acetylvinylthio) guanine and cis-6-(2-acetylvinylthio) purine case less in vivo Toxicity than 6-Thioguanine after Single and Multiple-Dose Regimens. Mol. Cancer Therapeutics. 2002. 1: p. 1211-1220.

1. A method for inhibiting replication of an RNA virus comprising contacting said RNA virus with a replication-inhibiting amount of a compound having the formula:

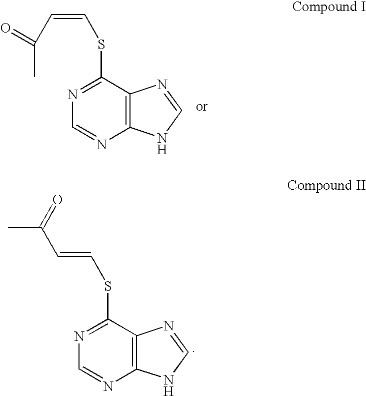

2. A method according to claim 1, wherein said RNA virus is a flavivirus.

3. A method according to claim 2, wherein said flavivirus is hepatitis C virus (HCV) or bovine diarrhea virus (BVDV).

4. A method according to claim 1, wherein said contacting occurs in vivo.

5. A method according to claim 1, wherein said contacting occurs in a glutathione rich cell or tissue.

6. A method according to claim 5 wherein said cell or tissue is liver, kidney or gastrointestinal tract tissue.

7. A method according to claim 1, wherein said compound (I) or (II) is associated with a targeting agent.

8. A method according to claim 1, wherein said compound further comprises a glycoside.

9. A method according to claim 1, wherein a derivative of said compound is a metabolite of compound (I) or (II).

10. A method according to claim 9, wherein said metabolite of compound (I) or (II) is 6-mercaptopurine.

11. A method for inhibiting replication of an RNA virus in a host comprising administering to a host in need thereof a pharmaceutical composition including a therapeutically effective amount of a compound having the formula:

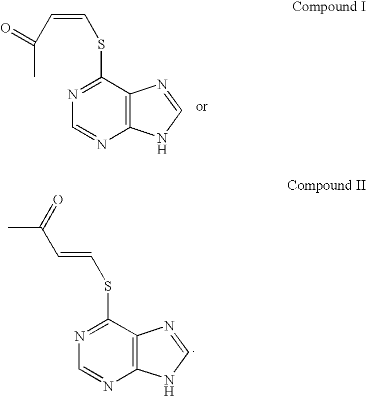

12. A method according to claim 11, wherein said RNA virus is a flavivirus.

13. A method according to claim 12, wherein said flavivirus is hepatitis C virus (HCV).

14. A method according to claim 11, wherein compound (I) or (II) is associated with a targeting agent.

15. A method according to claim 11, wherein said host is a liver transplant patient.

16. A method according to claim 11, wherein said compound further comprises a glycoside.

17. A method according to claim 11, wherein a derivative of said compound is a metabolite of compound (I) or (II).

18. A method according to claim 17, wherein said metabolite of compound (I) or (II) is 6-mercaptopurine.

19. A method according to claim 17, wherein said derivative possessing antiviral activity is associated with a targeting agent capable of targeting said derivative to a pre-selected cell or tissue.

20. A method according to claim 19 wherein said pre-selected cell or tissue is lung tissue.
